(19) United States
(12) Patent Application Publication
(10) Pub. No.: US 2025/0058006 A1
(43) Pub. Date: Feb. 20, 2025

(54) Hypochlorous Acid Disinfection Water Machine

(71) Applicants: CRAWFORD GLOBAL LIMITED, Hong Kong (CN); ETI SOLID STATE LIGHTING INC, Wheeling, IL (US); DERNIER AND HAMLYN INC., Wheeling, IL (US)

(72) Inventors: Donglei WANG, Zhuhai, Guangdong (CN); Li ZHANG, Zhuhai, Guangdong (CN); Songhong DING, Zhuhai, Guangdong (CN); Qunyong CAO, Zhuhai, Guangdong (CN); Rixin QUAN, Zhuhai, Guangdong (CN); Gang ZHAO, Zhuhai, Guangdong (CN); Qian LI, Zhuhai, Guangdong (CN)

(21) Appl. No.: 18/723,697

(22) PCT Filed: Dec. 24, 2021
(86) PCT No.: PCT/CN2021/141354

§ 371 Date: Jun. 24, 2024

(51) Int. Cl. A61L 2/18; A61L 2/24; A61L 2/26; A61L 101/06; C02F 1/00; C02F 1/461

(52) U.S. Cl. CPC A61L 2/18; A61L 2/24; A61L 2/26; C02F 1/008; C02F 1/46104; A61L 2101/06; A61L 2202/11; A61L 2202/14; C02F 2201/005; C02F 2201/4611; C02F 2201/46115; C02F 2201/46145; C02F 2201/4618; C02F 2209/02; C02F 2209/29; C02F 2209/40

(57) ABSTRACT

Provided is a hypochlorous acid disinfection water machine. The hypochlorous acid disinfection water machine includes a water inlet pipeline; a primary electrolyte cell, which is internally provided with a cation exchange membrane, and the outlet end of the water inlet pipeline communicates with the primary electrolyte cell; and a secondary electrolyte cell, which is a non-membrane electrolytic cell, a primary hypochlorous acid solution in an anode chamber of the primary electrolyte cell communicates with the secondary electrolyte cell, the primary hypochlorous acid solution in the anode chamber of the primary electrolyte cell is electrolyzed again in the secondary electrolyte cell to obtain a secondary hypochlorous acid solution. The hypochlorous acid disinfection water machine provided by the disclosure may solve the problem of poor concentration stability of a hypochlorous acid disinfection water machine in the relevant art.

### CROSS-REFERENCE TO RELATED APPLICATION

The disclosure is a National Stage Filing of the PCT International Application No. PCT/CN2021/141354 filed on Dec. 24, 2021, which is hereby incorporated by reference in its entirety.

### TECHNICAL FIELD

The disclosure relates to the technical field of disinfection devices, and in particular to a hypochlorous acid disinfection water machine.

### BACKGROUND

Hypochlorous acid solution is widely applied to work and life as disinfection liquid, and various hypochlorous acid water manufacturing machines are available on the market at present, most of which directly control an ion exchange diaphragm electrolytic cell or a non-membrane electrolytic cell to electrolyze a special electrolyte so as to generate hypochlorous acid water.

For hypochlorous acid in the hypochlorous acid water machine, the pH value in the hypochlorous acid water machine generally refers to the type of chlorine ion substance present in water, and when the pH value is 5-6.5, hypochlorous acid with basically pure chlorine ion substance is available. At present, a hypochlorous acid generator on the market generally performs electrolysis by a conventional electrolytic cell, and then generates hypochlorous acid with different concentrations by a special solution formula, such as adding vinegar or hydrochloric acid, with the pH value being 5-6.5, but the concentration of hypochlorous acid generated by the method is unstable.

However, in the existing common diaphragm electrolysis process, alkali may be mixed with chlorine gas and hypochlorous acid, and part of chlorine ions are occupied, resulting in a decrease in the concentration of hypochlorous acid formed inside the electrolytic cell.

As described above, the existing hypochlorous acid disinfection water machine has the problem of poor concentration stability when generating hypochlorous acid solution.

### SUMMARY

Some embodiments of the disclosure are to provide a hypochlorous acid disinfection water machine, for solving the problem of poor concentration stability of a hypochlorous acid disinfection water machine in the relevant art.

In an embodiment of the present disclosure, a hypochlorous acid disinfection water machine is provided. The hypochlorous acid disinfection water machine includes a water inlet pipeline; a primary electrolyte cell, a cation exchange membrane is disposed in the primary electrolyte cell, and an outlet end of the water inlet pipeline communicates with the primary electrolyte cell; and a secondary electrolyte cell, which is a non-membrane electrolytic cell, a primary hypochlorous acid solution in an anode chamber of the primary electrolyte cell communicates with the secondary electrolyte cell, the primary hypochlorous acid solution in the anode chamber of the primary electrolyte cell is electrolyzed again in the secondary electrolyte cell to obtain a secondary hypochlorous acid solution, and an acidity of the secondary hypochlorous acid solution is smaller than that of the primary hypochlorous acid solution.

In an embodiment, the hypochlorous acid disinfection water machine further includes an electrolyte storage part, which is configured to store electrolyte and provide electrolyte for the anode chamber of the primary electrolyte cell.

In an embodiment, the anode chamber of the primary electrolyte cell communicates with the secondary electrolyte cell through a communicating pipeline. The hypochlorous acid disinfection water machine further includes a concentration monitoring part, which is disposed on the communicating pipeline; and a controller, which is connected with the concentration monitoring part and a pump of the hypochlorous acid disinfection water machine, the pump is configured to convey NaCl or electrolyte solution, and the controller controls a working state of the pump according to a concentration of the primary hypochlorous acid solution collected by the concentration monitoring part so as to keep the concentration of the primary hypochlorous acid solution stable.

In an embodiment, the concentration monitoring part includes a flow sensor, the flow sensor is configured to monitor a flow of the primary hypochlorous acid solution in the communicating pipeline, and the controller adjusts a power of the pump according to a flow change condition monitored by the flow sensor so as to keep the concentration of the primary hypochlorous acid solution stable.

In an embodiment, the flow sensor has a preset flow Q1, a preset pump speed V1 and a preset minimum flow Q2, when an actual flow q is smaller than the preset minimum flow Q2, the hypochlorous acid disinfection water machine stops working and gives an alarm; when the actual flow q is greater than the preset minimum flow Q2 and smaller than the preset flow Q1, the hypochlorous acid disinfection water machine works normally, and an actual pump speed V2=q*V1/Q1; when the actual flow q is greater than the preset flow Q1, the hypochlorous acid disinfection water machine works normally, and the actual pump speed V2=q*V1/Q1; and when the actual flow q is equal to the preset flow Q1, the hypochlorous acid disinfection water machine works normally, and the actual pump speed V2=V1.

In an embodiment, the hypochlorous acid disinfection water machine further includes a controller, a pressure stabilizing part is disposed on the water inlet pipeline, and the controller is electrically connected with the pressure stabilizing part.

In an embodiment, the pressure stabilizing part is a pressure reducing valve.

In an embodiment, a pressure stabilizing part is disposed on the water inlet pipeline, a filtering structure is further disposed on the water inlet pipeline, and the filtering structure is located at the upstream position of the pressure stabilizing part.

In an embodiment, the hypochlorous acid disinfection water machine further includes a discharge pipeline, which communicates with a cathode chamber of the primary electrolyte cell; and/or a flow limiting valve is further disposed on the discharge pipeline.

In an embodiment, the hypochlorous acid disinfection water machine further includes an interactive part, which is connected with the controller through a signal, the controller is connected with the primary electrolyte cell and/or the secondary electrolyte cell through a signal, control programs of multiple concentration gears are preset in the controller, the interactive part selects a control program of the corresponding concentration gear according to an external instruction, and the controller controls the working states of the pump, the primary electrolyte cell and/or the secondary electrolyte cell.

In an embodiment, a pressure stabilizing part is disposed on the water inlet pipeline. The hypochlorous acid disinfection water machine further includes a controller; a discharge pipeline, and a flow limiting valve is further disposed on the discharge pipeline; a pump; and an interactive part, the interactive part is connected with the controller through a signal, the controller is connected with the pressure stabilizing part, the flow limiting valve, the primary electrolyte cell and the secondary electrolyte cell through a signal, control programs of multiple concentration gears are preset in the controller, the interactive part selects a control program of the corresponding concentration gear according to an external instruction, and in the different control programs, a current of the primary electrolyte cell, a current of the secondary electrolyte cell, a power of the pump, a set value of the pressure stabilizing part and a set value of the flow limiting valve are not exactly the same.

In an embodiment, the hypochlorous acid disinfection water machine further includes a concentration monitoring part, which is disposed on a communicating pipeline, and the controller is connected with the concentration monitoring part; and the interactive part further includes a display unit, and the display unit is configured to display at least one of a working state of the primary electrolyte cell, a working state of the secondary electrolyte cell, a working state of the pump, a working state of the concentration monitoring part, a working state of the pressure stabilizing part, and a working state of the flow limiting valve.

In an embodiment, the hypochlorous acid disinfection water machine further includes an electrolyte storage part. The hypochlorous acid disinfection water machine further includes a NaCl content detecting part, which is configured to detect NaCl concentration in the electrolyte storage part; and a temperature detecting part, which is configured to detect ambient temperature in the electrolyte storage part, and both the NaCl content detecting part and the temperature detecting part are electrically connected with the controller, and the display unit is configured to display NaCl concentration and/or ambient temperature.

In an embodiment, the hypochlorous acid disinfection water machine further includes an alarm part, which is electrically connected with the controller. When a concentration monitored by the concentration monitoring part exceeds a preset flow range, the controller controls the alarm part to give an alarm for prompt.

In an embodiment, the hypochlorous acid disinfection water machine further includes an alarm part, which is electrically connected with the controller. The display unit is configured to display alarm information of the alarm part, and the alarm information at least includes: fault information of the primary electrolyte cell, fault information of the secondary electrolyte cell, fault information of the concentration monitoring part, limit exceeding information of concentration range, fault information of the pump, water shortage prompt, NaCl shortage prompt and temperature alarm prompt.

In an embodiment, the interactive part further includes at least one of an on-off key, a mode selection key, a concentration gear selection key, a concentration adjustment key, and a capacity adjustment key connected to the controller through a signal.

In an embodiment, the hypochlorous acid disinfection water machine further includes a concentration monitoring part and a controller. The hypochlorous acid disinfection water machine further includes a shell; partition plates, the plurality of partition plates are disposed in the shell for separating inside space of the shell into a plurality of independent functional areas, the plurality of independent functional areas include an electrolysis functional area, a liquid storage functional area and an electric element placing area, and the primary electrolyte cell, the secondary electrolyte cell and the concentration monitoring part are disposed in the electrolysis functional area; and a liquid delivering pipeline, which extends into the electrolysis functional area from the liquid storage functional area, and the controller is disposed in the electric element placing area.

In an embodiment, the hypochlorous acid disinfection water machine further includes an electrolyte storage part, the plurality of independent functional areas further include a power supply compartment, and/or the electrolyte storage part is disposed in the liquid storage functional area.

In an embodiment, the power supply compartment is located at the top of the shell; the electric element placing area is located on the front side of the shell; the electrolysis functional area is located on the back side of the shell; and the liquid storage functional area extends from the front side of the shell to the back side of the shell, and the liquid storage functional area is disposed in parallel with an area formed by the electric element placing area and the electrolysis functional area.

In an embodiment, a back plate of the shell is detachably disposed for opening or closing the electrolysis functional area; and/or the hypochlorous acid disinfection water machine further includes an opening/closing door, which is disposed at an opening of the liquid storage functional area.

Some embodiments of the disclosure are to provide a hypochlorous acid disinfection water machine. The hypochlorous acid disinfection water machine includes a water inlet pipeline; a primary electrolyte cell, which is internally provided with an ion exchange membrane, and an outlet end of the water inlet pipeline communicates with the primary electrolyte cell; an electrolyte solution storage part, which is configured to store NaCl solution, the electrolyte solution storage part communicates with the primary electrolyte cell through a liquid delivering pipeline, and a pump is further disposed on the liquid delivering pipeline; a secondary electrolyte cell, which is a non-membrane electrolytic cell, a primary hypochlorous acid solution in a cation cell of the primary electrolyte cell communicates with the secondary electrolyte cell through a communicating pipeline, the primary hypochlorous acid solution in the cation cell of the primary electrolyte cell is electrolyzed again in the secondary electrolyte cell to obtain a secondary hypochlorous acid solution, and an acidity of the secondary hypochlorous acid solution is smaller than that of the primary hypochlorous acid solution; a concentration monitoring part, which is disposed on the communicating pipeline; and a controller, which is connected with the concentration monitoring part and the pump, and the controller controls a working state of the pump according to a concentration of the primary hypochlorous acid solution collected by the concentration monitoring part so as to keep the concentration of the primary hypochlorous acid solution stable.

By adoption of the technical solution of the disclosure, water inside the water inlet pipeline and electrolyte solution inside the electrolyte storage part both enter the primary electrolyte cell to be mixed, after mixing, electrolysis reaction occurs inside the primary electrolyte cell, so that for liquid after electrolysis, under action of the cation exchange membrane, chlorine ions with negative charges enter the anode chamber to have oxidation reaction to form hypochlorous acid solution, sodium ions enter the cathode chamber to have reduction reaction, namely, the primary hypochlorous acid solution formed after electrolysis enters the anode chamber, the hypochlorous acid solution inside the anode chamber enters the non-membrane secondary electrolytic cell to be subjected to secondary electrolysis reaction to obtain secondary hypochlorous acid solution.

In addition, in the disclosure, the concentration monitoring part is disposed between the secondary electrolyte cell and the primary electrolyte cell for monitoring primary hypochlorous acid solution, meanwhile, the controller controls the working state of the pump according to the concentration of the primary hypochlorous acid solution monitored by the concentration monitoring part, so that the primary hypochlorous acid solution produced through electrolysis reaction by electrolyte solution and water inside the water inlet pipeline after entering the primary electrolyte cell has a stable concentration, then phenomenon that the concentration of the secondary hypochlorous acid solution and target concentration are different due to change of liquid inlet amount of the water inlet pipeline is avoided, and the stability of the concentration of the secondary hypochlorous acid solution is improved. In the solution, under the control of the controller, the concentration of the primary hypochlorous acid solution may be monitored in real time to improve the intelligence of the hypochlorous acid disinfection water machine, secondary hypochlorous acid solution of secondary electrolysis may be obtained only if a user provides electrolyte solution, and the secondary hypochlorous acid solution obtained through secondary electrolysis does not need to be processed, so that the operation is simple and convenient.

### BRIEF DESCRIPTION OF THE DRAWINGS

The drawings forming a part of the disclosure in the specification are adopted to provide a further understanding to the disclosure. Schematic embodiments of the disclosure and descriptions thereof are adopted to explain the disclosure and not intended to form improper limits to the disclosure. In the drawings:

FIG. 1 illustrates a block diagram of a work process of a hypochlorous acid disinfection water machine provided by the disclosure.

FIG. 2 illustrates a schematic structure diagram of a three-dimensional view of one hypochlorous acid disinfection water machine provided by the disclosure.

FIG. 3 illustrates a schematic structure diagram of a three-dimensional view of another hypochlorous acid disinfection water machine provided by the disclosure.

The drawings include the following reference signs.

100. Shell; 110. Back plate; 120. Liquid storage functional area; 130. Electrolysis functional area; 140. Ventilation opening; 150. Outgoing connector; 160. First liquid inlet connector; 170. Discharge connector; 180. Power supply compartment; 200. Door body; 300. Interactive part; 400. Electrolyte storage part; 500. Discharge pipeline; 600. Filtering structure; 700. Pressure stabilizing part; 800. Electromagnetic valve; 900. Electrolyte solution; 1010. Pump; 1020. Primary electrolyte cell; 1030. Concentration monitoring part; 1040. Secondary electrolyte cell; 1050. Primary hypochlorous acid solution; 1060. Secondary hypochlorous acid solution; and 1070. Flow limiting valve.

### DETAILED DESCRIPTION OF THE EMBODIMENTS

It is to be noted that the embodiments and features in the embodiments of the disclosure may be combined with each other without conflict. The disclosure will be described in detail below with reference to the accompanying drawings and the embodiments.

It is to be noted that, unless defined otherwise, all technical and scientific terms used herein have the same meaning as commonly understood by those of ordinary skill in the art to which this disclosure belongs.

In the disclosure, unless stated to the contrary, the orientation term such as “upper, lower, top, bottom” or the like, generally refers to the direction shown in the drawings, or to the vertical, perpendicular, or gravitational direction of components; likewise, for ease of understanding and description, “inner and outer” refer to inner and outer relative to the profile of the components, but the above orientation terms are not intended to limit the disclosure.

### Embodiment 1

The disclosure provides a hypochlorous acid disinfection water machine, for solving the problem of poor concentration stability of a hypochlorous acid disinfection water machine in the relevant art. The hypochlorous acid disinfection water machine may be applied to household field or industrial field.

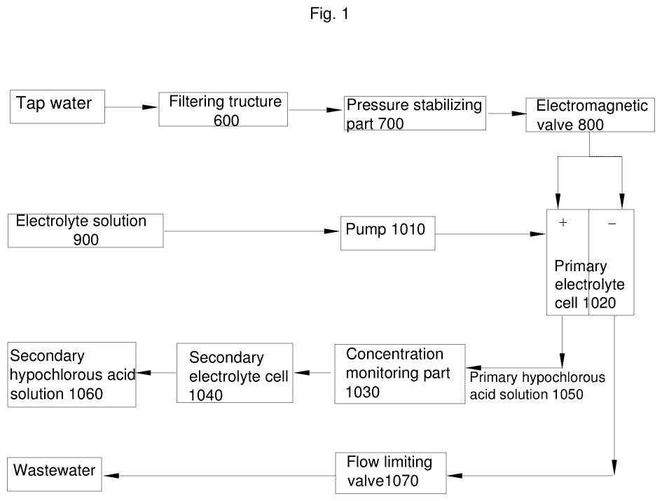

As shown in FIG. 1, a hypochlorous acid disinfection water machine includes a water inlet pipeline, a primary electrolyte cell 1020, an electrolyte solution storage part 400, a secondary electrolyte cell 1040, a concentration monitoring part 1030 and a controller, an cation exchange membrane is disposed in the primary electrolyte cell 1020, and an outlet end of the water inlet pipeline communicates with the primary electrolyte cell 1020; the electrolyte solution storage part 400 is configured to store NaCl or NaCl solution, namely, electrolyte solution, the electrolyte solution storage part 400 communicates with the primary electrolyte cell 1020 through a liquid delivering pipeline, and a pump 1010 is further disposed on the liquid delivering pipeline; the secondary electrolyte cell 1040 is a non-membrane electrolytic cell, a primary hypochlorous acid solution 1050 in an anode chamber of the primary electrolyte cell 1020 communicates with the secondary electrolyte cell 1040 through a communicating pipeline, the primary hypochlorous acid solution 1050 in the anode chamber of the primary electrolyte cell 1020 is electrolyzed again in the secondary electrolyte cell to obtain a secondary hypochlorous acid solution 1060, and an acidity of the secondary hypochlorous acid solution 1060 is smaller than that of the primary hypochlorous acid solution 1050; the concentration monitoring part 1030 is disposed on the communicating pipeline; and the controller is connected with the concentration monitoring part 1030 and the pump 1010, and the controller controls a working state of the pump 1010 according to a concentration of the primary hypochlorous acid solution 1050 collected by the concentration monitoring part 1030 so as to keep the concentration of the primary hypochlorous acid solution 1050 stable.

Specifically, water inside the water inlet pipeline and electrolyte solution 900 inside the electrolyte storage part 400 both enter the primary electrolyte cell 1020 to be mixed, after mixing, electrolysis reaction occurs inside the primary electrolyte cell 1020, so that for liquid after electrolysis, under action of the cation exchange membrane, chlorine ions with negative charges enter the anode chamber to have oxidation reaction to form hypochlorous acid solution, sodium ions enter the cathode chamber to have reduction reaction, namely, the primary hypochlorous acid solution 1050 formed after electrolysis enters the anode chamber, the hypochlorous acid solution inside the anode chamber enters the non-membrane secondary electrolyte cell to be subjected to secondary electrolysis reaction to obtain secondary hypochlorous acid solution 1060.

Of course, in the embodiment, the electrolyte is not limited to NaCl or NaCl solution, electrolytes of other types may also be adopted, specifically to achieve electrolysis and produce hypochlorous acid.

In addition, in the disclosure, the concentration monitoring part 1030 is disposed between the secondary electrolyte cell and the primary electrolyte cell 1020 for monitoring primary hypochlorous acid solution, meanwhile, the controller controls the working state of the pump 1010 according to the concentration of the primary hypochlorous acid solution monitored by the concentration monitoring part 1030, so that the primary hypochlorous acid solution 1050 produced through electrolysis reaction by electrolyte solution 900 and water inside the water inlet pipeline after entering the primary electrolyte cell 1020 has a stable concentration, then phenomenon that the concentration of the secondary hypochlorous acid solution 1060 and target concentration are different due to change of liquid inlet amount of the water inlet pipeline is avoided, and the stability of the concentration of the secondary hypochlorous acid solution 1060 is improved. In the solution, under the control of the controller, the concentration of the primary hypochlorous acid solution 1050 may be monitored in real time to improve the intelligence of the hypochlorous acid disinfection water machine, secondary hypochlorous acid solution 1060 of secondary electrolysis may be obtained only if a user provides electrolyte solution 900, and the secondary hypochlorous acid solution 1060 obtained through secondary electrolysis does not need to be processed, so that the operation is simple and convenient.

In an embodiment, inside the primary electrolyte cell 1020, with the cation exchange membrane as a boundary, the primary electrolyte cell 1020 is separated into a cathode chamber and an anode chamber, after electrolysis reaction occurs inside the primary electrolyte cell 1020, primary hypochlorous acid solution 1050 is formed inside the anode chamber and leads to the secondary electrolyte cell 1040, sodium hydroxide solution is formed inside the cathode chamber, which is discharged through a discharge pipeline 500 to be collected as wastewater, a flow limiting valve 1070 is disposed on the discharge pipeline 500 to realize on-off of the discharge pipeline 500 and control liquid discharge speed, and by controlling flow inside the discharge pipeline 500, a mixing ratio between the electrolyte solution 900 and water inside the primary electrolyte cell 1020.

The primary hypochlorous acid solution 1050 is pure hypochlorous acid solution, the pH value of the pure hypochlorous acid solution is 3-4, and the pH value of sodium hydroxide solution is 9-10.

In the embodiment, hypochlorous acid solution is produced by electrolysis of NaCl solution, a user only needs to provide NaCl or NaCl solution, a salt bottle is disposed, the salt bottle is configured to place NaCl or NaCl solution, and the salt bottle communicates with the primary electrolyte cell 1020 through the pump 1010 for introducing NaCl or NaCl solution to the inside of the primary electrolyte cell 1020.

In an embodiment, the user may put NaCl crystals and NaCl powder into the salt bottle, so that NaCl crystals and NaCl powder enter the primary electrolyte cell 1020 to have electrolysis reaction inside the primary electrolyte cell 1020; and the user may also pour NaCl solution into the salt bottle, so that the NaCl solution is fed into the primary electrolyte cell 1020 to have electrolysis reaction inside the primary electrolyte cell 1020.

Of course, the specific placing form may be adaptively adjusted according to demands of the user.

In the primary electrolyte cell 1020, water and electrolyte solution 900 are mainly electrolyzed, in the secondary electrolyte cell 1040, primary hypochlorous acid solution 1050 is mainly subjected to secondary electrolysis to produce secondary hypochlorous acid solution 1060. A acidity of secondary hypochlorous acid solution 1060 is reduced, and good oxidation and sterilization effects are achieved.

It is to be noted that the controller may be electrically connected with the pressure stabilizing part 700, the flow limiting valve 1070, the primary electrolyte cell 1020 and the secondary electrolyte cell 1040 at the same time, and controls the current of the primary electrolyte cell 1020, the current of the secondary electrolyte cell 1040, the power of the pump 1010, the working state of the pressure stabilizing part 700, a working state of the flow limiting valve 1070 and a power of the pump 1010 according to a required concentration of the secondary hypochlorous acid solution 1060. Under the control of the controller, the current of the primary electrolyte cell 1020, the current of the secondary electrolyte cell 1040, the power of the pump 1010, the working state of the pressure stabilizing part 700, the working state of the flow limiting valve 1070 and the power of the pump 1010 acts in coordination to ensure concentration of the secondary hypochlorous acid solution 1060.

In the disclosure, the pump 1010 is a peristaltic pump, and a power and a speed of the peristaltic pump are set through an electronic control program.

As shown in FIG. 1, the concentration monitoring part 1030 includes a flow sensor, the flow sensor is configured to monitor the flow of the primary hypochlorous acid solution 1050 in the communicating pipeline, and the controller adjusts a power of the pump 1010 according to the flow change condition monitored by the flow sensor so as to keep the concentration of the primary hypochlorous acid solution 1050 stable.

In an embodiment, the flow sensor monitors the flow of the primary hypochlorous acid solution 1050, when the flow of the primary hypochlorous acid solution 1050 increases, it means that excessively liquid inside the water inlet pipeline flows into the primary electrolyte cell 1020, resulting in a decrease of the concentration of the primary hypochlorous acid solution 1050 formed inside the primary electrolyte cell 1020, in such a case, the controller controls the power of the pump 1010 to increase the flow of the electrolyte solution 900, so as to balance the flows of the electrolyte solution 900 and the liquid inlet pipe, so that the primary hypochlorous acid solution 1050 produced inside the primary electrolyte cell 1020 flows to the secondary electrolyte cell 1040 in a stable concentration state.

Of course, a concentration monitoring part 1030 may also be disposed between the primary electrolyte cell 1020 and the secondary electrolyte cell 1040 for monitoring a concentration of the primary hypochlorous acid solution 1050, and the controller sends an electric signal to the pump 1010 for adjusting the flow of the electrolyte solution 900, so that the concentration of the primary hypochlorous acid solution 1050 is stable.

In the embodiment, the flow sensor has a preset flow Q1, a preset pump speed V1 and a preset minimum flow Q2. When an actual flow q is smaller than the preset minimum flow Q2, the hypochlorous acid disinfection water machine stops working and gives an alarm; when the actual flow q is greater than the preset minimum flow Q2 and smaller than the preset flow Q1, the hypochlorous acid disinfection water machine works normally, and an actual pump speed V2=q*V1/Q1; when the actual flow q is greater than the preset flow Q1, the hypochlorous acid disinfection water machine works normally, and the actual pump speed V2=q*V1/Q1; and when the actual flow q is equal to the preset flow Q1, the hypochlorous acid disinfection water machine works normally, and the actual pump speed V2=V1.

In an embodiment, the flow sensor detects actual flow q, flowing to the secondary electrolyte cell 1040, of the primary hypochlorous acid solution 1050 produced in the primary electrolyte cell 1020, when the actual flow q is smaller than the preset minimum flow Q2, the inside of the primary electrolyte cell 1020 is short of liquid, and the hypochlorous acid disinfection water machine stops working and gives an alarm.

In an embodiment, as described above, the actual flow q is positively correlated with the actual pump speed V2, when the actual flow q is too large or too small during the normal operation of the hypochlorous acid disinfection water machine, the actual flow q may be adjusted by adjusting the actual pump speed V2, so that the actual flow q is equal to the preset flow Q1, and primary hypochlorous acid solution 1050 and secondary hypochlorous acid solution 1060 with target concentrations are prepared.

It is to be noted that secondary hypochlorous acid solutions 1060 of different concentrations may be obtained according to actual demands of a user, specifically, a concentration value of secondary hypochlorous acid solution of the required concentration may be set on the controller, in some embodiments, the concentration may be 98%, 95%, 70%, etc.

Three different implementation modes are provided in the embodiment according to different structural parts controlled by the controller, specifically as follows.

In a specific implementation mode not illustrated, the controller is connected with the primary electrolyte cell 1020 and controls the current of the primary electrolyte cell 1020 according to the required concentration of the secondary hypochlorous acid solutions 1060 in order to produce the secondary hypochlorous acid solution 1060 with a preset concentration by controlling the concentration of chlorine ions produced in the primary hypochlorous acid solution 1050.

Specifically, by controlling current in the primary electrolyte cell 1020, the electrolysis reaction inside the primary electrolyte cell 1020 is adjusted, and by controlling the amount of chlorine ions produced through electrolysis reaction, the concentration of the primary hypochlorous acid solution 1050 is adjusted.

In another specific implementation mode not illustrated, the controller is connected with the secondary electrolyte cell 1040 and controls the current of the secondary electrolyte cell 1040 according to the required concentration of the secondary hypochlorous acid solutions 1060 in order to produce the secondary hypochlorous acid solution 1060 with a preset concentration by controlling the concentration of chlorine ions produced in the secondary hypochlorous acid solution 1060.

In another specific implementation mode not illustrated, the controller is electrically connected with the pump 1010, by controlling the power of the pump 1010, the power of the pump 1010 is adjusted, the flow of the electrolyte solution 900 is adjusted by different powers, so that the concentration of chlorine ions is adjusted for controlling the concentration of the secondary hypochlorous acid solution 1060. Of course, it also may be that the controller controls the water inlet pipeline, and the concentration of chlorine ions is adjusted by adjusting the flow of the water inlet pipeline, so that the secondary hypochlorous acid solution 1060 with preset concentration is produced.

Specifically, the pump 1010 outputs different rotation speeds by controlling power of the pump 1010, and the flow of the electrolyte solution 900 is adjusted through different rotation speeds, so as to ensure concentration of primary hypochlorous acid solution 1050 inside the primary electrolyte cell 1020.

As shown in FIG. 1, a pressure stabilizing part 700 is disposed on the water inlet pipeline, and the controller is electrically connected with the pressure stabilizing part 700. The controller is electrically connected with the pressure stabilizing part 700 for adjusting the flow of liquid in the water inlet pipeline, so as to realize the primary electrolyte cell 1020 with stable flow of liquid.

Specifically, the pressure stabilizing part 700 is a pressure valve, the controller controls the pressure valve to realize regulation on liquid inside the water inlet pipeline, namely, realize pressure stabilizing effect, meanwhile, the flow may also be adjusted by adjusting the pressure stabilizing part 700, in an embodiment, flow of liquid inside the water inlet pipeline is increased by pressurization, so that the concentration of electrolyte inside the primary electrolyte cell 1020 is reduced to produce chlorine ions with low concentration after primary electrolysis reaction, then the concentration of the secondary hypochlorous acid solution 1060 is also reduced, on the contrary, the flow of liquid inside the water inlet pipeline may be reduced through the pressure stabilizing part 700, so that the concentration of the secondary hypochlorous acid solution 1060 is increased, the principle is the same, and no elaboration will be made here.

The pressure valve may realize an effect of protection on the water inlet pipeline, so phenomenon of water leakage may be avoided, then when pressure of liquid in the water inlet pipeline is too large, the pressure in the water inlet pipeline may be controlled within a safe range, in some embodiments, 0.2-0.3 MPa, by adjusting the pressure valve.

In an embodiment, an electromagnetic valve 800 is disposed between the pressure stabilizing part 700 and the primary electrolyte cell 1020, so that on-off of the water inlet pipeline may be controlled by controlling the electromagnetic valve 800, then it may be realized that only when primary electrolysis reaction needs to be carried out in the primary electrolyte cell, the controller sends an electric signal to the electromagnetic valve 800, the electromagnetic valve 800 is in an open state, and liquid in the water inlet pipeline enters the primary electrolyte cell 1020. Of course, the flow of liquid in the water inlet pipeline may also be adjusted by controlling the electromagnetic valve 800, and then concentrations of the primary hypochlorous acid solution 1050 and secondary hypochlorous acid solution 1060 may be controlled.

As shown in FIG. 1, a filtering structure 600 is further disposed on the water inlet pipeline, and the filtering structure 600 is located at the upstream position of the pressure stabilizing part 700.

Specifically, a liquid inlet of the water inlet pipeline communicates with a switch of tap water, and the filtering structure 600 is disposed on the water inlet pipeline for filtering tap water, so as to prevent impurities in tap water from entering the primary electrolyte cell 1020 to affect primary electrolysis reaction and further affect the production of the primary hypochlorous acid solution 1050.

Of course, the liquid inlet of the water inlet pipeline may also communicate with other structures or devices providing liquid, and the filtering structure 600 may realize filtering on liquid inside the water inlet pipeline.

In an embodiment, the filtering structure 600 may remove impurities such as calcium and magnesium ions in water, so as to reduce influence on electrolysis by substances such as calcium and magnesium ions attaching on an electrolysis sheet, meanwhile, the protection effect on the electrolysis sheet is realized, and further, the service life of the electrolysis sheet is prolonged.

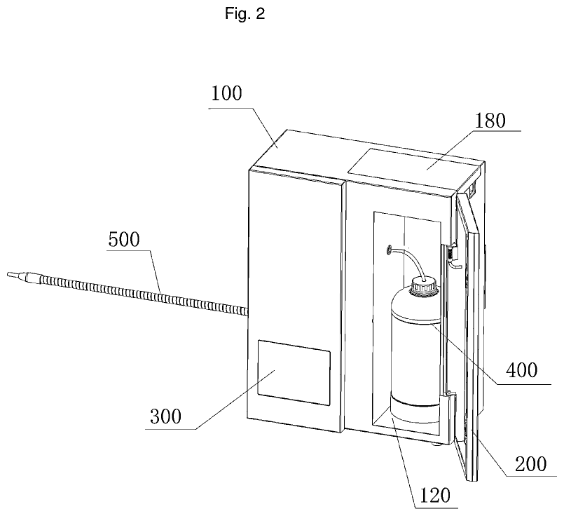

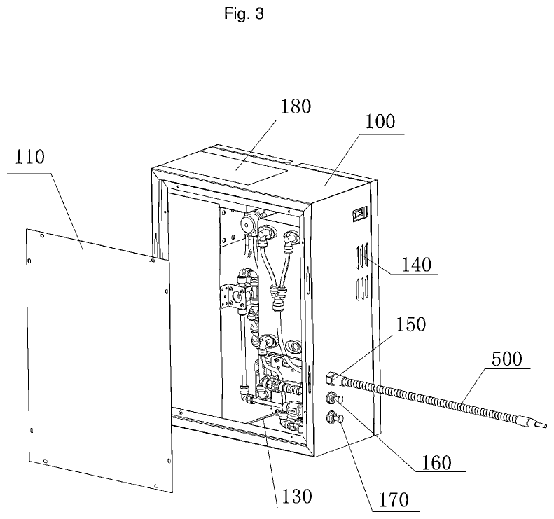

As shown in FIGS. 2-3, the hypochlorous acid disinfection water machine further includes an interactive part 300, the interactive part 300 is connected with the controller through a signal, control programs of multiple concentration gears are preset in the controller, the interactive part 300 selects the control program of the corresponding concentration gear according to an external instruction, and the controller controls the working states of the pump 1010, the primary electrolyte cell 1020 and/or the secondary electrolyte cell 1040.

Specifically, in different control programs, the current of the primary electrolyte cell 1020, the current of the secondary electrolyte cell 1040, the power of the pump 1010, a set value of the pressure stabilizing part 700 and a set value of the flow limiting valve 1070 are not exactly the same. Pressure stabilization is not essential to overall stability, the flow limiting valve mainly control discharge speed of wastewater, and a pressure limiting valve may affect the overall concentration, but is not essential.

In the embodiment, the controller has 8 different concentration gears, for producing 8 secondary hypochlorous acid solutions 1060 of different concentrations, and then hypochlorous acid solutions of different concentrations may be met.

In an embodiment, the interactive part 300 further includes a display unit and an operation part, and the display unit is configured to display at least one of the working state of the primary electrolyte cell 1020, the working state of the secondary electrolyte cell 1040, the working state of the pump 1010, the working state of the concentration monitoring part 1030, the working state of the pressure stabilizing part 700, and the working state of the flow limiting valve 1070. In an embodiment, the display unit may display the working state of the primary electrolyte cell 1020, the working state of the secondary electrolyte cell 1040, the working state of the pump 1010, the working state of the concentration monitoring part 1030, the working state of the pressure stabilizing part 700, and the working state of the flow limiting valve 1070 at the same time, so that the working states of the primary electrolyte cell, the secondary electrolyte cell, the pump 1010, the pressure stabilizing part 700, the concentration monitoring part 1030 and the flow limiting valve 1070 may be observed and known in real time.

In an embodiment, the operation part at least includes at least one of an on-off key, a mode selection key, a concentration gear selection key, a concentration adjustment key, and a capacity adjustment key, the on-off key controls start and stop of the interactive part 300, the interactive part 300 may be controlled to be switched between a working state and a standby state by short press of the on-off key, when the interactive part 300 is controlled to be in a working state, the hypochlorous acid disinfection water machine is started for producing water, and the display unit displays the volume and concentration of the secondary hypochlorous acid solution 1060; and when the interactive part 300 is controlled to be in a standby state, the hypochlorous acid disinfection water machine stops producing water, and the display unit does not display data. When the interactive part 300 is in a standby state, the mode selection key is pressed for switching between controlling the concentration of the secondary hypochlorous acid solution 1060 and controlling the volume of the secondary hypochlorous acid solution 1060, when it is switched to control the concentration of the secondary hypochlorous acid solution 1060, the concentration of the secondary hypochlorous acid solution 1060 may be adjusted and controlled, and when it is switched to control the volume of the secondary hypochlorous acid solution 1060, the volume of the secondary hypochlorous acid solution 1060 may be controlled. A plurality of concentration gear selection keys are disposed, and each concentration gear selection key corresponds to one preset concentration value, in some embodiments, 50 ppm, 100 ppm, 150 ppm and 200 ppm correspond to four secondary hypochlorous acid solutions 1060 of different concentrations. The concentration adjustment keys include a concentration increase key and a concentration decrease key, similarly, the capacity adjustment keys include a volume increase key and a volume decrease key, and it is to be noted that the hypochlorous acid disinfection water machine stops working when the volume reaches a preset value.

In the embodiment, the hypochlorous acid disinfection water machine further includes a NaCl content detecting part and a temperature detecting part, the NaCl content detecting part is configured to detect NaCl concentration in the electrolyte storage part 400, the temperature detecting part is configured to detect ambient temperature in the electrolyte storage part 400, both the NaCl content detecting part and the temperature detecting part are electrically connected with the controller, and the display unit is configured to display NaCl concentration and ambient temperature.

By disposing the NaCl content detecting part and display on the display unit, a user is assisted in controlling the amount of NaCl, so that the electrolyte solution 900 reaches the standard, the user gradually adds NaCl under display and reminding of the display unit according to requirements, so that the accuracy of adding NaCl is improved, and then the phenomenon that the concentration of the primary hypochlorous acid solution 1050 produced through electrolysis reaction in the primary electrolyte cell 1020 is unstable due to excessive or insufficient adding of NaCl may be avoided.

By disposing the temperature detecting part, ambient temperature in the electrolyte storage part 400 may be monitored in real time, and when the temperature is abnormal, it may be known by the user in time.

In the embodiment, the hypochlorous acid disinfection water machine further includes an alarm part, which is electrically connected with the controller, when the flow monitored by the flow sensor exceeds a preset flow range, the controller controls the alarm part to give an alarm for prompt. When the flow monitored by the flow sensor exceeds a preset flow range, the concentration of the produced secondary hypochlorous acid solution 1060 is lower than a preset concentration, when an alarm gives an alarm for prompting that the flow monitored by the flow sensor exceeds the preset flow range, the flow of the pump 1010 or the water inlet pipeline needs to be adjusted so that the flow monitored by the flow sensor is within the preset flow range, and the concentration of the secondary hypochlorous acid solution 1060 is ensured to be within the preset range.

The display unit is configured to display alarm information of the alarm part, and the alarm information at least includes: fault information of the primary electrolyte cell 1020, fault information of the secondary electrolyte cell 1040, fault information of the concentration monitoring part 1030, limit exceeding information of concentration range, fault information of the pump 1010, water shortage prompt, NaCl shortage prompt and temperature alarm prompt. By displaying alarm information on the display unit, the user may intuitively observe and know where a specific fault occurs, and adjust according to the specific fault, thus improving the accuracy and efficiency of fault handling.

When there is water shortage, the flow sensor detects that the flow does not change within a certain period of time, and the flow is lower than preset flow, indicating that no water inlet pipeline is connected or the flow inside the water inlet pipeline is too low, the device stops working, a buzzer makes a “Didi” sound, a fault prompt code E1 is displayed, the on-off key or the mode selection key may be pressed for clearing the alarm, so that the device enters a standby state, and detecting starts again when it is switched to the working state from the standby state.

When there is shortage of NaCl, the NaCl content detecting part detects shortage of NaCl, the device stops working, the buzzer makes a “Didi” sound, a fault prompt code E2 is displayed, the on-off key or the mode selection key may be pressed for clearing the alarm, so that the device enters a standby state, and detecting starts again when it is switched to the working state from the standby state.

When circuit fault occurs on the primary electrolyte cell 1020 and the secondary electrolyte cell 1040, the device stops working, the buzzer makes a “Didi” sound, a fault prompt code E3 is displayed, the on-off key or the mode selection key may be pressed for clearing the alarm, so that the device enters a standby state, and detecting starts again when it is switched to the working state from the standby state.

When the temperature is too high, the temperature detecting part detects that the temperature increases abnormally, in an embodiment, when the temperature exceeds 85° C., the device stops working, the buzzer makes a “Didi” sound, a fault prompt code E4 is displayed, the on-off key or the mode selection key may be pressed for clearing the alarm, so that the device enters a standby state, and detecting starts again when it is switched to the working state from the standby state.

When the temperature detecting part fails, the device stops working, the buzzer makes a “Didi” sound, a fault prompt code E5 is displayed, the on-off key or the mode selection key may be pressed for clearing the alarm, so that the device enters a standby state, and detecting starts again when it is switched to the working state from the standby state.

As shown in FIGS. 2-3, the hypochlorous acid disinfection water machine further includes a shell 100, partition plates, the plurality of partition plates are disposed in the shell 100 for separating inside space of the shell 100 into a plurality of independent functional areas, and the plurality of independent functional areas include an electrolysis functional area 130, a liquid storage functional area 120 and an electric element placing area. The electrolyte storage part 400 is disposed in the liquid storage functional area 120; the primary electrolyte cell 1020, the secondary electrolyte cell and the concentration monitoring part 1030 are disposed in the electrolysis functional area 130; a liquid delivering pipeline extends into the electrolysis functional area 130 from the liquid storage functional area 120, and the controller is disposed in the electric element placing area.

Specifically, the shell 100 plays the role of protection, the shell 100 is separated into a plurality of independent functional areas through the partition plates to realize water and electricity separation, so that the safety of use is improved, meanwhile, the space utilization rate inside the shell 100 is improved by partition through the partition plates, when components of the electrolysis functional area 130 need to be adjusted, only space where the electrolysis functional area 130 is located needs to be adjusted without affecting other spaces, the electric element placing area and the electrolysis functional area 130 are disposed in isolation, the electrolysis functional area 130, the liquid storage functional area 120 and the electric element placing area are sealed and isolated from each other to realize water and electricity separation, so that the safety of use is improved, so that the phenomenon of electricity leakage caused by influence of liquid on electric appliances is avoided, and meanwhile, the aesthetic feeling is also improved.

In some embodiments, the plurality of independent functional areas further include a power supply compartment 180, a cover plate is disposed on the power supply compartment, and operation on a power supply inside the power supply compartment 180 is realized by assembling and dissembling the cover plate. The power supply is independently closed in the power supply compartment 180, the power supply may provide electricity for the electric appliance inside the shell 100, and the power supply independently disposed is convenient to replace and maintain.

In the embodiment, an opening/closing door which is movably disposed is disposed on a front face plate of the shell 100, the opening/closing door is hinged with the front face plate, and the interactive part 300 is embedded on the opening/closing door or the front face plate, so as to facilitate operation and penetration of the user.

An outgoing connector 150, a first liquid inlet connector 160 and a discharge connector 170 are disposed on the side of the shell 100, the secondary electrolyte cell 1040 communicates with the outgoing connector 150 for discharging produced secondary hypochlorous acid solution 1060 to a liquid storage pipeline through the outgoing connector 150 for collection; and a first liquid inlet is configured to provide tap water for the hypochlorous acid disinfection water machine, and the discharge connector 170 communicates with the cathode chamber of the primary electrolyte cell 1020 for discharging sodium hydroxide liquid in the cathode chamber.

In an embodiment, a plurality of ventilation openings 140 are further disposed on the shell 100 for ventilation and heat dissipation.

As shown in FIGS. 2-3, the power supply compartment 180 is located at the top of the shell 100, the electric element placing area is located on the front side of the shell 100, the electrolysis functional area 130 is located on the back side of the shell 100, the liquid storage functional area 120 extends from the front side of the shell 100 to the back side of the shell 100, and the liquid storage functional area 120 is disposed in parallel with an area formed by the electric element placing area and the electrolysis functional area 130.

Specifically, the shell 100 is separated into left and right spaces, the liquid storage functional area 120 is located in one of the left space and the right space, the liquid storage functional area 120 is provided with a groove in an extending manner from the front side to the back side, an electrolyte storage part 400, namely, a salt bottle, is placed in the liquid storage functional area 120, and when the corresponding opening/closing door is opened, electrolyte solution 900 may be added to the electrolyte storage part 400; and the other of the left space and the right space is separated by a partition into an electric element placing area on the front area and an electrolysis functional area 130 on the back area, and the interactive part 300 is disposed on a front face plate corresponding to the electric element placing area or the opening/closing door.

In an embodiment, the back plate 110 is detachably disposed for opening or closing the electrolysis functional area 130, so that devices in the electrolysis functional area 130 inside the shell 100 may be conveniently operated and detected.

During electrolysis process in the primary electrolyte cell 1020, the anode main reaction:

2Cl⁻−2e⁻→Cl₂⬆;

Cl₂+H₂O→HCl+HClO;

### Cathode Main Reaction:

2H₂O+2e⁻→H₂⬆+2OH⁻.

During electrolysis, 2Cl⁻ loses electrons to produce chlorine gas, which dissolves in water and reacts with it to produce hydrochloric acid and hypochlorous acid, which exist inside the anode chamber. Due to presence of Na⁺ and Cl⁻ in NaCl solution, Na⁺ moves into the cathode chamber and forms NaOH with OH⁻ in the cathode chamber, and NaOH is discharged by a discharge pipeline.

Due to use of a cation membrane electrolytic cell, in the process of electrolysis, only Na⁺ is allowed to pass the membrane, Cl⁻ and OH⁻ are blocked, so that the problem of mixing of alkali with Cl₂ and HClO in the process of electrolysis of a common diaphragm method is solved, therefore, acidic HClO solution may be obtained in the anode chamber of the primary electrolyte cell and NaOH solution may be obtained in the cathode chamber.

During electrolysis process in the secondary electrolyte cell 1040, the anode reaction:

2Cl⁻−2e⁻→Cl₂⬆;

Cl₂+H₂O→HCl+HClO;

### Cathode Reaction:

2H₂O+2e⁻→H₂⬆+2OH⁻.

Through secondary electrolysis, the acidity of primary hypochlorous acid solution may be reduced, meanwhile, the concentration of available chlorine may be improved, and secondary hypochlorous acid solution is produced.

### Embodiment 2

The disclosure provides a hypochlorous acid disinfection water machine, for solving the problem of poor concentration stability of a hypochlorous acid disinfection water machine in the relevant art. The hypochlorous acid disinfection water machine may be applied to household field or industrial field.

In the embodiment, the hypochlorous acid disinfection water machine includes a water inlet pipeline, a primary electrolyte cell 1020, an electrolyte solution storage part, a secondary electrolyte cell 1040, a concentration monitoring part 1030 and a controller, an ion exchange membrane is disposed in the primary electrolyte cell 1020, and an outlet end of the water inlet pipeline communicates with the primary electrolyte cell 1020; the electrolyte solution storage part is configured to store NaCl solution, the electrolyte solution storage part communicates with the primary electrolyte cell 1020 through a liquid delivering pipeline, and a pump 1010 is further disposed on the liquid delivering pipeline; the secondary electrolyte cell 1040 is a non-membrane electrolytic cell, a primary hypochlorous acid solution 1050 in a cation cell of the primary electrolyte cell 1020 communicates with the secondary electrolyte cell 1040 through a communicating pipeline, the primary hypochlorous acid solution 1050 in the cation cell of the primary electrolyte cell 1020 is electrolyzed again in the secondary electrolyte cell to obtain a secondary hypochlorous acid solution 1060, and an acidity of the secondary hypochlorous acid solution 1060 is smaller than that of the primary hypochlorous acid solution 1050; the concentration monitoring part 1030 is disposed on the communicating pipeline; and the controller is connected with the concentration monitoring part 1030 and the pump 1010, and the controller controls a working state of the pump 1010 according to a concentration of the primary hypochlorous acid solution 1050 collected by the concentration monitoring part 1030 so as to keep the concentration of the primary hypochlorous acid solution 1050 stable.

In an embodiment the concentration monitoring part 1030 is disposed between the secondary electrolyte cell and the primary electrolyte cell 1020 for monitoring primary hypochlorous acid, meanwhile, the controller controls the working state of the pump 1010 according to the concentration of the primary hypochlorous acid monitored by the concentration monitoring part 1030, so that the primary hypochlorous acid solution 1050 produced through electrolysis reaction by electrolyte solution 900 and water inside the water inlet pipeline after entering the primary electrolyte cell 1020 has a stable concentration, then the phenomenon that the concentration of the secondary hypochlorous acid solution 1060 and target concentration are different due to change of liquid inlet amount of the water inlet pipeline is avoided, and the stability of the concentration of the secondary hypochlorous acid solution 1060 is improved. In the solution, under the control of the controller, the concentration of the primary hypochlorous acid solution 1050 may be monitored in real time to improve the intelligence of the hypochlorous acid disinfection water machine, secondary hypochlorous acid solution 1060 of secondary electrolysis may be obtained only if a user provides electrolyte solution 900, and the secondary hypochlorous acid solution 1060 obtained through secondary electrolysis does not need to be processed, so that the operation is simple and convenient.

Obviously, the above-described embodiments are only some, but not all, embodiments of the disclosure. All other embodiments obtained by those of ordinary skill in the art based on the embodiments of the disclosure without creative efforts shall fall within the protection scope of the disclosure.

It is to be noted that terms used herein are for the purpose of describing specific implementation modes only and are not intended to be limiting of exemplary implementation modes according to the disclosure. Unless otherwise directed by the context, singular forms of terms used herein are intended to include plural forms. Besides, it will be also appreciated that when terms “contain” and/or “include” are used in the description, it is indicated that features, steps, operations, devices, assemblies and/or a combination thereof exist.

It is to be noted that the terms “first,” “second,” and the like in the specification, the claims and the drawings of the disclosure are configured for distinguishing between similar objects and not necessarily for describing a particular sequence or sequential order. It will be appreciated that such data may be interchangeable where appropriate, so that the implementation modes of the disclosure described herein can be implemented in a sequence except for those illustrated or described herein.

The foregoing is merely preferred embodiments of the disclosure and is not intended to limit the disclosure, and various modifications and variations of the disclosure may be available for those skilled in the art. Any modifications, equivalent replacements, improvements and the like made within the spirit and principle of the disclosure shall fall within the scope of protection of the disclosure.

1. A hypochlorous acid disinfection water machine, comprising:
a water inlet pipeline;
a primary electrolyte cell, a cation exchange membrane is disposed in the primary electrolyte cell, and an outlet end of the water inlet pipeline communicates with the primary electrolyte cell;
a secondary electrolyte cell, the secondary electrolyte cell is a non-membrane electrolytic cell, a primary hypochlorous acid solution in an anode chamber of the primary electrolyte cell communicates with the secondary electrolyte cell, the primary hypochlorous acid solution in the anode chamber of the primary electrolyte cell is electrolyzed again in the secondary electrolytic cell to obtain a secondary hypochlorous acid solution, and an acidity of the secondary hypochlorous acid solution is smaller than that of the primary hypochlorous acid solution.

2. The hypochlorous acid disinfection water machine according to claim 1, wherein the hypochlorous acid disinfection water machine further comprises an electrolyte storage part, which is configured to store electrolyte, and the electrolyte storage part is configured to provide electrolyte for the anode chamber of the primary electrolyte cell.

3. The hypochlorous acid disinfection water machine according to claim 1, wherein the anode chamber of the primary electrolyte cell communicates with the secondary electrolyte cell through a communicating pipeline, the hypochlorous acid disinfection water machine further comprises:
a concentration monitoring part, the concentration monitoring part is disposed on the communicating pipeline;
a controller, which is connected with the concentration monitoring part, the controller is connected with a pump of the hypochlorous acid disinfection water machine, the pump is configured to convey NaCl or electrolyte solution, and the controller controls a working state of the pump according to a concentration of the primary hypochlorous acid solution collected by the concentration monitoring part so as to keep the concentration of the primary hypochlorous acid solution stable.

4. The hypochlorous acid disinfection water machine according to claim 3, wherein the concentration monitoring part comprises a flow sensor, the flow sensor is configured to monitor a flow of the primary hypochlorous acid solution in the communicating pipeline, and the controller adjusts a power of the pump according to a flow change condition monitored by the flow sensor so as to keep the concentration of the primary hypochlorous acid solution stable.

5. The hypochlorous acid disinfection water machine according to claim 4, wherein the flow sensor has a preset flow Q1, a preset pump speed V1 and a preset minimum flow Q2, and
when an actual flow q is smaller than the preset minimum flow Q2, the hypochlorous acid disinfection water machine stops working and gives an alarm;
when the actual flow q is greater than the preset minimum flow Q2 and smaller than the preset flow Q1, the hypochlorous acid disinfection water machine works normally, and an actual pump speed V2=q*V1/Q1;
when the actual flow q is greater than the preset flow Q1, the hypochlorous acid disinfection water machine works normally, and the actual pump speed V2=q*V1/Q1;
when the actual flow q is equal to the preset flow Q1, the hypochlorous acid disinfection water machine works normally, and the actual pump speed V2=V1.

6. The hypochlorous acid disinfection water machine according to claim 1, wherein the hypochlorous acid disinfection water machine further comprises a controller, a pressure stabilizing part is disposed on the water inlet pipeline, and the controller is electrically connected with the pressure stabilizing part, the pressure stabilizing part is a pressure reducing valve.

7. (canceled)

8. The hypochlorous acid disinfection water machine according to claim 1, wherein a pressure stabilizing part is disposed on the water inlet pipeline, a filtering structure is further disposed on the water inlet pipeline, and the filtering structure is located at the upstream position of the pressure stabilizing part.

9. The hypochlorous acid disinfection water machine according to claim 1, wherein the hypochlorous acid disinfection water machine further comprises a discharge pipeline,
the discharge pipeline communicates with a cathode chamber of the primary electrolyte cell; and/or
a flow limiting valve is further disposed on the discharge pipeline.

10. The hypochlorous acid disinfection water machine according to claim 3, wherein the hypochlorous acid disinfection water machine further comprises an interactive part, the interactive part is connected with the controller through a signal, the controller is connected with the primary electrolyte cell and/or the secondary electrolyte cell through a signal, control programs of multiple concentration gears are preset in the controller, the interactive part selects a control program of the corresponding concentration gear according to an external instruction, and the controller controls the working states of the pump, the primary electrolyte cell and/or the secondary electrolyte cell.

11. The hypochlorous acid disinfection water machine according to claim 1, wherein a pressure stabilizing part is disposed on the water inlet pipeline, the hypochlorous acid disinfection water machine further comprises:
a controller;
a discharge pipeline, and a flow limiting valve is further disposed on the discharge pipeline;
a pump;
an interactive part, the interactive part is connected with the controller through a signal, the controller is connected with the pressure stabilizing part, the flow limiting valve, the primary electrolyte cell and the secondary electrolyte cell through a signal, control programs of multiple concentration gears are preset in the controller, the interactive part selects a control program of the corresponding concentration gear according to an external instruction, and in the different control programs, a current of the primary electrolyte cell, a current of the secondary electrolyte cell, a power of the pump, a set value of the pressure stabilizing part and a set value of the flow limiting valve are not exactly the same.

12. The hypochlorous acid disinfection water machine according to claim 11, wherein the hypochlorous acid disinfection water machine further comprises a concentration monitoring part, the concentration monitoring part is disposed on a communicating pipeline, and the controller is connected with the concentration monitoring part;
the interactive part further comprises a display unit, and the display unit is configured to display at least one of a working state of the primary electrolyte cell, a working state of the secondary electrolyte cell, a working state of the pump, a working state of the concentration monitoring part, a working state of the pressure stabilizing part, and a working state of the flow limiting valve.

13. The hypochlorous acid disinfection water machine according to claim 12, wherein the hypochlorous acid disinfection water machine further comprises an electrolyte storage part, and further comprises:
a NaCl content detecting part, which is configured to detect NaCl concentration in the electrolyte storage part;
a temperature detecting part, which is configured to detect ambient temperature in the electrolyte storage part;
both the NaCl content detecting part and the temperature detecting part are electrically connected with the controller, and the display unit is configured to display the NaCl concentration and/or the ambient temperature.

14. The hypochlorous acid disinfection water machine according to claim 3, wherein the hypochlorous acid disinfection water machine further comprises an alarm part, which is electrically connected with the controller, and when a concentration monitored by the concentration monitoring part exceeds a preset flow range, the controller controls the alarm part to give an alarm for prompt.

15. The hypochlorous acid disinfection water machine according to claim 13, wherein the hypochlorous acid disinfection water machine further comprises an alarm part, which is electrically connected with the controller, the display unit is configured to display alarm information of the alarm part, and the alarm information at least comprises: fault information of the primary electrolyte cell, fault information of the secondary electrolyte cell, fault information of the concentration monitoring part, limit exceeding information of concentration range, fault information of the pump, water shortage prompt, NaCl shortage prompt and temperature alarm prompt.

16. The hypochlorous acid disinfection water machine according to claim 11, wherein the interactive part further comprises at least one of an on-off key, a mode selection key, a concentration gear selection key, a concentration adjustment key, and a capacity adjustment key connected to the controller through a signal.

17. The hypochlorous acid disinfection water machine according to any of claim 1, wherein the hypochlorous acid disinfection water machine comprises a concentration monitoring part and a controller, and the hypochlorous acid disinfection water machine further comprises:
a shell;
partition plates, the plurality of partition plates are disposed in the shell for separating inside space of the shell into a plurality of independent functional areas, and the plurality of independent functional areas comprise an electrolysis functional area, a liquid storage functional area and an electric element placing area, and
the primary electrolyte cell, the secondary electrolyte cell and the concentration monitoring part are disposed in the electrolysis functional area;
a liquid delivering pipeline, which extends into the electrolysis functional area from the liquid storage functional area;
the controller is disposed in the electric element placing area.

18. The hypochlorous acid disinfection water machine according to claim 17, wherein the hypochlorous acid disinfection water machine further comprises an electrolyte storage part, and the plurality of independent functional areas further comprise:
a power supply compartment; and/or
the electrolyte storage part is disposed in the liquid storage functional area; and/or
the electrolysis functional area, the liquid storage functional area and the electric element placing area are sealed and isolated from each other.

19. The hypochlorous acid disinfection water machine according to claim 18, wherein
the power supply compartment is located at the top of the shell;
the electric element placing area is located on the front side of the shell;
the electrolysis functional area is located on the back side of the shell;
the liquid storage functional area extends from the front side of the shell to the back side of the shell, and the liquid storage functional area is disposed in parallel with
an area formed by the electric element placing area and the electrolysis functional area.

20. The hypochlorous acid disinfection water machine according to claim 17, wherein
a back plate of the shell is detachably disposed for opening or closing the electrolysis functional area; and/or
the hypochlorous acid disinfection water machine further comprises an opening/closing door, which is disposed at an opening of the liquid storage functional area.

21. A hypochlorous acid disinfection water machine, comprising:
a water inlet pipeline;
a primary electrolyte cell, an ion exchange membrane is disposed in the primary electrolyte cell, and an outlet end of the water inlet pipeline communicates with the primary electrolyte cell;
an electrolyte solution storage part, which is configured to store NaCl solution, the electrolyte solution storage part communicates with the primary electrolyte cell through a liquid delivering pipeline, and a pump is further disposed on the liquid delivering pipeline;
a secondary electrolyte cell, the secondary electrolyte cell is a non-membrane electrolytic cell, a primary hypochlorous acid solution in a cation cell of the primary electrolyte cell communicates with the secondary electrolyte cell through a communicating pipeline, the primary hypochlorous acid solution in the cation cell of the primary electrolyte cell is electrolyzed again in the secondary electrolyte cell to obtain a secondary hypochlorous acid solution, and an acidity of the secondary hypochlorous acid solution is smaller than that of the primary hypochlorous acid solution;
a concentration monitoring part, and the concentration monitoring part is disposed on the communicating pipeline;
a controller, which is connected with the concentration monitoring part, the controller is connected with the pump, and the controller controls a working state of the pump according to a concentration of the primary hypochlorous acid solution collected by the concentration monitoring part so as to keep the concentration of the primary hypochlorous acid solution stable.
